(19) United States
(12) Patent Application Publication
(10) Pub. No.: US 2026/0053826 A1
(43) Pub. Date: Feb. 26, 2026

(54) FACE AND BODY OIL COMPOSITION

(71) Applicant: Heather Harris, Gardena, CA (US)

(72) Inventor: Heather Harris, Gardena, CA (US)

(21) Appl. No.: 18/811,246

(22) Filed: Aug. 21, 2024

(51) Int. Cl. A61K 31/00; A61K 31/10; A61K 31/165; A61K 31/375; A61K 31/385; A61K 31/4415; A61K 31/51; A61K 31/714; A61K 33/06; A61K 33/242; A61K 36/28; A61K 36/324; A61K 36/61; A61K 36/67; A61K 36/9066; A61K 36/9068; A61K 47/20

(52) U.S. Cl. CPC A61K 31/658; A61K 31/10; A61K 31/165; A61K 31/375; A61K 31/385; A61K 31/4415; A61K 31/51; A61K 31/714; A61K 33/06; A61K 33/242; A61K 36/28; A61K 36/324; A61K 36/61; A61K 36/67; A61K 36/9066; A61K 36/9068; A61K 47/20

(57) ABSTRACT

A body and face oil composition is disclosed. The composition includes a sufficient amount of Dimethyl sulfoxide (DMSO), a sufficient amount of Cannabidiol (CBD), a sufficient amount of gold, a sufficient amount of one or more vitamins, and a sufficient amount of one or more minerals. The composition, when administered to a human at an effective dose, is beneficial for reducing pain, for regenerating nerves, skin, bone, and tissue, and for anti-aging.

### TECHNICAL FIELD

The embodiments presented herein generally relate to face and body oil compositions and more specifically relate to face and body oil compositions which provide nerve pain relief, regenerative properties, and anti-aging properties.

### BACKGROUND

Oils are commonly used in skincare products and medicines as a carrier for various essential oils and compounds. Depending on the use, a base oil (i.e., carrier oil) is mixed with ingredients which provide benefits such as anti-aging properties, pain relief, regenerative properties (such as providing skin, bone, hair, and/or nerve regeneration) among other benefits for general health and well-being.

### SUMMARY OF THE INVENTION

This summary is provided to introduce a variety of concepts in a simplified form that is disclosed further in the detailed description of the embodiments. This summary is not intended for determining or limiting the scope of the claimed subject matter.

The embodiments provided herein disclosed a body and face oil composition. The composition includes a sufficient amount of Dimethyl sulfoxide (DMSO), a sufficient amount of Cannabidiol (CBD), a sufficient amount of gold, a sufficient amount of one or more vitamins, and a sufficient amount of one or more minerals. The composition, when administered to a human at an effective dose, is beneficial for reducing pain, for regenerating nerves, skin, bone, and tissue, and for anti-aging.

In one aspect, the composition includes 10% DMSO which is added to the oil and mixed.

In one aspect, the sufficient amount of CBD is about 10 grams for each effective dose.

In one aspect, the one or more vitamins includes a sufficient amount of at least one of the following: Vitamin B1, Vitamin B6, Vitamin B12, and Vitamin C.

In one aspect, the composition is further comprised of at least one of the following: sunflower oil, coconut oil, and coconut glycerin.

In one aspect, the sufficient amount of Cannabidiol is provided as Cannabidiol Distillate and Cannabidiol Isolate.

In one aspect, the composition is further comprised of at least one of the following: ginger, cloves, feverfew flowers, chamomile flowers, magnesium chloride, capsaicin powder, white turmeric powder, pepper corns, Alpha Lipoic Acid (ALA) powder, MSM powder; and Boswellia Extract.

During production, the composition is formulated including amounts of ingredients which provide the aforementioned benefits. In a large sterile jar 12 oz of organic sunflower oil and 4 oz or organic coconut oil are added. Next, ¼ cup of organic chamomile flowers and 1/4 cup of organic feverfew flowers is added to the jar which is then sealed a lid and placed in a sunny area for 2 weeks. A sunny area is defined as an environment which received a consistent and regular amount of light from the sun.

Next, 12 oz or organic sunflower oil and 4 oz of organic coconut oil is added to a second sterile jar. ½ cup of freshly grated organic ginger and ¼ cup of ground organic cloves is then added to the second jar. Let rest in a cool dark place for 2 weeks, such that sunlight or other light sources do not emit light to the jar and its contents.

In a third sterile jar, 8 oz of organic coconut glycerin and ¼ cup of organic dried cayenne pepper are added and allowed to rest for 2 weeks. Once ready to use, the contents are strained.

Once the first, second, and third jars are rested for the aforementioned time periods, 16 oz of chamomile and feverfew oil along with 16 oz of ginger and clove oil along with ¼ cup of capsaicin (Cayenne) oil are added.

Next, 37 grams of Organic CBD Isolate is added followed by ¼ cup of magnesium chloride. Next, 2 tablespoons of organic white turmeric powder and 1 tablespoon of organic pepper corns are added to the oil. A lid is then used to seal the mixture which is then heated in a canner at 200° Fahrenheit for 10 hours. Once complete, 10 grams CBD Distillate is added to the oil.

Next, 1 tbs. Organic Boswellia Extract, 1 tbs. MSM powder, 1 tbs. Organic vitamin C powder, 1 tsp. Vitamin B1, Vitamin B6, and Vitamin B12, and 1 tbs. ALA Powder are added, and the lid is replaced. The contents sealed in the jar are then heated at 180° Fahrenheit for 4-hours. Following heating, the contents are strained twice using a cheese cloth or similar straining mechanism.

Next, 20 mg of 24 Karat Gold (in an edible format) are added, and the lid is replaced. The contents are then heated at 180° Fahrenheit for 2-hours and then allowed to cool. Once cool, The lid is replaced, and contents are heated at 180*F degrees for 2 hours. 10% DMSO is then added to the oil (3.4 oz) and the contents are mixed. The composition is now ready to be packaged (i.e., bottled).

Other objects and advantages of the various embodiments will become obvious and intuitive to the reader, and it is intended that these objects and advantages are within the scope of the present embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing in detail exemplary embodiments, it is noted that the embodiments reside primarily in combinations of components related to particular devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In general, the embodiments provided here relate to a face and body oil composition. The composition is formulated to reduce, prevent, and/or inhibit nerve pain as well as to aid in the regeneration of the nerves, skin, tissue, and bone as well as to provide anti-aging properties. The composition is comprised of Cannabidiol (CBD), Dimethyl sulfoxide (DMSO), gold, one or more vitamins and one or more minerals.

In some embodiments, the composition is comprised of a sufficient amount of CBD. CBD is notable for its benefits related to preventing, inhibiting the progression of, and/or treating various neurological disorders and has been attributed to aiding in pain relief and various other therapeutic benefits.

In some embodiments, the composition is comprised of a sufficient amount of DMSO. DMSO is known to penetrate the skin and other membranes without damaging them to provide a carrier mechanism for other compounds into a biological system (i.e., the human body). DMSO is also known to be utilized as a topical analgesic, a vehicle for the application of pharmaceuticals (such as a transdermal drug delivery systems), as an anti-inflammatory, and as an antioxidant. In such, the inclusion of DMSO may aid in reducing nerve pain (via its anti-inflammatory properties) as well as a means for delivering other compounds via its transdermal delivery capabilities.

In some embodiments, DMSO is utilized as a non-toxic solvent that increases the body's ability to absorb other compounds.

The combination of CBD and DMSO may provide synergistic benefits to the user, especially when applied to the skin for the purposes of regenerating nerves, tissues, skin, and bone as well as for the purposes of relieving pain associated with nerve damage, damage to the skin, tissue damage, and bone damage.

In some embodiments, CBD when combined with DMSO allows the skin to absorb the CBD significantly faster when compared to compounds containing CBD alone. DMSO may also facilitate the penetration of the CBD into the body which increases its effects.

In some embodiments, the composition includes a sufficient amount of gold (e.g., 24-karat gold). Gold is known to aid in reducing inflammation. Further, gold may also provide and enhance antibacterial and antioxidant properties when utilized in the present composition.

In some embodiments, the composition includes a sufficient amount of a vitamin blend comprised of one or more vitamins.

In some embodiments, the composition includes a sufficient amount of a mineral blend comprised of one or more minerals.

In some embodiments, the composition may provide a warming sensation when applied to the user's skin.

In some embodiments, the composition is comprised of sufficient amounts of each of the following ingredients: Organic Cold Pressed Sunflower Oil, Organic Virgin Cold Pressed Coconut Oil, Organic Coconut Glycerin, Organic CBD Isolate, Organic CBD Distillate, Organic Ginger, Organic Cloves, Organic Feverfew Flowers, Organic Chamomile Flowers, Magnesium Chloride, DMSO (Pharmaceutical Grade), Organic Dried Cayenne Pepper (Capsaicin Powder), Organic White Turmeric Powder, Organic Pepper Corns, Alpha Lipoic Acid Powder (ALA), MSM Powder, Thiamine HCL Powder (Vitamin B1), Pyridoxine Powder (Vitamin B6), Vitamin B12, Organic Boswellia Extract, Organic Vitamin C Powder, and 24 Karat Edible Gold.

One skilled in the arts while particular ingredients are described above, the ingredients may not need to be organic, or may be different analogs, versions, etc. when compared to the aforementioned ingredients.

During production, the composition is formulated including amounts of ingredients which provide the aforementioned benefits. Sufficient amounts for each ingredient are provided in the below description. However, one skilled in the arts will readily understand that the specific amounts may be modified without departing from the scope of the embodiments described herein. In a large sterile jar 12 oz of organic sunflower oil and 4 oz or organic coconut oil are added. Next, ¼ cup of organic chamomile flowers and 1/4 cup of organic feverfew flowers is added to the jar which is then sealed a lid and placed in a sunny area for 2 weeks. A sunny area is defined as an environment which received a consistent and regular amount of light from the sun.

Next, 12 oz or organic sunflower oil and 4 oz of organic coconut oil is added to a second sterile jar. ½ cup of freshly grated organic ginger and ¼ cup of ground organic cloves is then added to the second jar. Let rest in a cool dark place for 2 weeks, such that sunlight or other light sources do not emit light to the jar and its contents.

In a third sterile jar, 8 oz of organic coconut glycerin and ¼ cup of organic dried cayenne pepper are added and allowed to rest for 2 weeks. Once ready to use, the contents are strained.

Once the first, second, and third jars are rested for the aforementioned time periods, 16 oz of chamomile and feverfew oil along with 16 oz of ginger and clove oil along with ¼ cup of capsaicin (Cayenne) oil are added.

Next, 37 grams of Organic CBD Isolate is added followed by ¼ cup of magnesium chloride. Next, 2 tablespoons of organic white turmeric powder and 1 tablespoon of organic pepper corns are added to the oil. A lid is then used to seal the mixture which is then heated in a canner at 200°Fahrenheit for 10 hours. Once complete, 10 grams CBD Distillate is added to the oil.

Next, 1 tbs. Organic Boswellia Extract, 1 tbs. MSM powder, 1 tbs. Organic vitamin C powder, 1 tsp. Vitamin B1, Vitamin B6, and Vitamin B12, and 1 tbs. ALA Powder are added, and the lid is replaced. The contents sealed in the jar are then heated at 180° Fahrenheit for 4-hours. Following heating, the contents are strained twice using a cheese cloth or similar straining mechanism.

Next, 20 mg of 24 Karat Gold (in an edible format) are added, and the lid is replaced. The contents are then heated at 180° Fahrenheit for 2-hours and then allowed to cool. Once cool, The lid is replaced, and contents are heated at 180*F degrees for 2 hours. 10% DMSO is then added to the oil (3.4 oz) and the contents are mixed. The composition is now ready to be packaged (i.e., bottled).

In some embodiments, one or more of the ingredients of the composition may be organic. Further, one or more of the ingredients may be pharmaceutical grade.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety to the extent allowed by applicable law and regulations. The systems and methods described herein may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

Many different embodiments have been disclosed herein. It will be understood that it would be unduly repetitious and obfuscating to literally describe every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

The foregoing is provided for purposes of explaining and describing embodiments of this disclosure. Modifications and adaptations to these embodiments will be apparent to those skilled in the art and may be made without departing from the scope or spirit of this disclosure.

As used herein and in the appended claims, the singular forms “a”, “an”, and “the” include plural referents unless the context clearly dictates otherwise.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

In many instances entities are described herein as being coupled to other entities. It should be understood that the terms “coupled” and “connected” (or any of their forms) are used interchangeably herein and, in both cases, are generic to the direct coupling of two entities (without any non-negligible (e.g., parasitic) intervening entities) and the indirect coupling of two entities (with one or more non-negligible intervening entities). Where entities are shown as being directly coupled together or described as coupled together without description of any intervening entity, it should be understood that those entities can be indirectly coupled together as well unless the context clearly dictates otherwise.

While the embodiments are susceptible to various modifications and alternative forms, specific examples thereof may or may not have been described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described herein. A variety of modifications and variations are possible in light of the above teachings without departing from the following claims.

What is claimed is:

1. A body and face oil composition, comprising:
a sufficient amount of Dimethyl sulfoxide (DMSO);
a sufficient amount of Cannabidiol (CBD);
a sufficient amount of gold;
a sufficient amount of one or more vitamins; and
a sufficient amount of one or more minerals,
wherein the composition, when administered to a human at an effective dose, is beneficial for reducing pain, for regenerating nerves, skin, bone, and tissue, and for anti-aging.

2. The composition of claim 1, wherein the sufficient amount of DMSO is about 10% DMSO.

3. The composition of claim 1, wherein the sufficient amount of CBD is about 10 grams for each effective dose.

4. The composition of claim 1, wherein the sufficient amount of DMSO enables the rapid absorption of the sufficient amount of CBD into the skin.

5. The composition of claim 1, wherein the sufficient amount of gold is 24 karat edible gold.

6. The composition of claim 1, wherein the sufficient amount of gold is 20mg.

7. The composition of claim 1, wherein the one or more vitamins includes a sufficient amount of at least one of the following: Vitamin B1, Vitamin B6, Vitamin B12, and Vitamin C.

8. The composition of claim 1, wherein the composition is further comprised of at least one of the following: sunflower oil, coconut oil, and coconut glycerin.

9. The composition of claim 1, wherein the sufficient amount of Cannabidiol is provided is Cannabidiol Distillate and Cannabidiol Isolate.

10. The composition of claim 1, further comprising a sufficient amount of each of the following:
ginger;
cloves;
feverfew flowers;
chamomile flowers;
magnesium chloride;
capsaicin powder;
white turmeric powder;
pepper corns;
Alpha Lipoic Acid (ALA) powder;
MSM powder; and
Boswellia Extract.

11. A body and face oil composition, comprising:
a sufficient amount of 10% Dimethyl sulfoxide (DMSO);
a sufficient amount of Cannabidiol (CBD);
a sufficient amount of 24 karat edible gold;
a sufficient amount of each of the following: Vitamin B1, Vitamin B6, Vitamin C, and Vitamin B12; and
a sufficient amount of one or more minerals,
wherein the composition, when administered to a human at an effective dose, is beneficial for reducing pain, for regenerating nerves, skin, bone, and tissue, and for anti-aging.

12. The composition of claim 11, wherein the sufficient amount of CBD is about 10 grams for each effective dose.

13. The composition of claim 12, wherein the composition is further comprised of 8 oz of sunflower oil, coconut oil, and coconut glycerin.

14. The composition of claim 13, wherein the sufficient amount of Cannabidiol is provided is 37 grams of Cannabidiol Distillate and 37 grams of Cannabidiol Isolate.

15. The composition of claim 14, wherein the sufficient amount of gold is 20mg.

16. The composition of claim 15, further comprising a sufficient amount of each of the following:

16. oz of ginger and clove oil;

16. oz of feverfew flower oil and chamomile flower oil;
1/4 cup of magnesium chloride;
1/4 cup of capsaicin powder;

2. tablespoons of white turmeric powder;

1. tablespoon of pepper corns;

1. tablespoon of Alpha Lipoic Acid (ALA) powder;

1. tablespoon of MSM powder; and

1. tablespoon of Boswellia Extract.

17. The composition of claim 16, wherein the composition is applied to the face or to the body to provide pain relief and to regenerate nerves, skin, tissue, and bone.
